(19) United States
(12) Patent Application Publication
(10) Pub. No.: US 2023/0056029 A1
(43) Pub. Date: Feb. 23, 2023

(54) BIO SIGNAL MEASURING DEVICE AND BIO SIGNAL IMAGING DEVICE AND BRAIN IMAGING BASED BRAIN DISEASE DIAGNOSTIC SYSTEM

(71) Applicant: SOLETOP CO., LTD., Daejeon (KR)

(72) Inventors: Yeongbo Sagong, Daejeon (KR); Sumin Jang, Daejeon (KR)

(21) Appl. No.: 17/412,600

(22) Filed: Aug. 26, 2021

(30) Foreign Application Priority Data
Aug. 23, 2021 (KR) 10-2021-0110971

(51) Int. Cl. A61B 5/026; A61B 5/00; A61B 5/11

(52) U.S. Cl. CPC A61B 5/0261; A61B 5/6814; A61B 5/7228; A61B 5/0042; A61B 5/0013; A61B 5/0015; A61B 5/742; A61B 5/1117; A61B 5/4088; A61B 2560/0406

(57) ABSTRACT

Provided is a biosignal measuring device 100 capable of easily calculating data on blood flow volume, blood flow velocity, and path length in the subject P as data for the time domain, and simplifying brain disease diagnosis based on this. It relates to a biosignal imaging device 1 and a brain image-based brain disease diagnosis system. To this end, the biosignal measuring apparatus 100 detects the reflected light signal after the light irradiated from the plurality of light irradiation units 111 and the plurality of light irradiation units 111 for irradiating light to the subject P are reflected. Based on the light signal detected by the measurement unit 110 including a plurality of light receiving units 112 and the light irradiation control unit 121 for controlling the light signal irradiated from each light irradiation unit 111 and the light receiving unit 112 and a calculation unit 120 including a signal processing unit 122 that calculates data for the subject P in the time domain.

### CROSS-REFERENCE TO RELATED APPLICATION

This application claims priority to and the benefit of Korean Patent Application No. 10-2021-0110971, filed on Aug. 23, 2021, in the Korean Intellectual Property Office, the entire content of which are incorporated herein by reference.

### FIELD OF THE INVENTION

The present invention relates to a biosignal measuring device 100, a biosignal imaging device 1, and a brain image-based brain disease diagnosis system, and more specifically, to a blood flow rate, blood flow velocity, and path length in a subject P. The present invention relates to a bio-signal measuring device 100, a bio-signal imaging device 1, and a brain image-based brain disease diagnosis system capable of easily calculating data as data for the time domain and simplifying brain disease diagnosis based on this.

### DESCRIPTION OF THE RELATED ART

In general, brain imaging-based brain disease diagnosis is used in Alzheimer's disease (including dementia), central nervous system inflammation, dopaminergic nervous system, dyskinesias, epilepsy and mental disorders (depression, schizophrenia, etc.) and brain tumor diseases.

The number of dementia patients in Korea is expected to exceed 1 million by 2024. In addition, the market size for the diagnosis of Alzheimer's disease, Parkinson's disease, etc. is judged to be steadily increasing. Accordingly, there is a demand for technological development of brain disease diagnosis based on brain imaging.

### SUMMARY OF THE INVENTION

According to a preferred embodiment for achieving the object of the present invention described above, a plurality of light irradiation units 111 for irradiating light to the subject P according to the present invention, and a plurality of light irradiation units 111 irradiated from the a measurement unit 110 including a plurality of light receiving units 112 for detecting the reflected light signal after the light is reflected; and a light irradiation control unit 121 for controlling the light signal irradiated from each light irradiation unit 111, and data on the subject P based on the light signal detected by the light receiving unit 112 in the time domain. a plurality of light beams such that the light irradiator 111 and the light receiver 112 are alternately disposed at the outermost portion of the measurement unit 110; The irradiation unit 111 and the plurality of light receiving units 112 are alternately arranged in a grid shape, and an imaginary line connecting each light receiving unit 112 located at the outermost portion of the measurement unit 110 forms a rectangular shape, and the rectangular shape In the four vertices, the light receiving unit 112 is disposed, and the light irradiation control unit 121 sequentially irradiates light modulated in response to three or more wavelength signals based on one light irradiation unit 111 with a time difference. Each light irradiator 111 is controlled so as to be possible, and in one light irradiator 111, light modulated in response to three or more wavelength signals is sequentially irradiated with a time difference, and one light irradiator 111 is used as a reference. In at least three light receiving units 112 adjacent to After demodulating the optical signal in time series corresponding to the preset position of the light irradiator 111, the demodulated signal is restored to the form of the wavelength signal before modulation, and based on the form of the restored wavelength signal, the blood flow volume, blood flow velocity, and path length are calculated as data for the time domain.

Hereinafter, the light irradiation control unit 121 includes: an optical characterization unit 1211 for selecting the three or more wavelength signals; an optical modulator 1212 for generating three or more modulated signals by modulating the three or more wavelength signals selected by the optical characterization unit 1211 with different frequencies; and a light control unit 1213 for controlling the time the light is irradiated from each light irradiation unit 111 so that three or more modulated signals are sequentially irradiated with a time difference based on one light irradiation unit 111; The signal processing unit 122 includes: a signal demodulator 1221 for generating demodulated signals by demodulating the optical signals respectively detected by the at least three light receiving units 112 in time series corresponding to the preset positions of the light emitting units 111; a signal restoration unit 1222 for restoring each demodulated signal generated by the signal demodulation unit 1221 to the form of a wavelength signal before modulation; a signal operation unit 1223 for generating an operation signal by calculating the shape of the wavelength signal restored by the signal restoration unit 1222; and calculating the blood flow volume, blood flow velocity, and path length in the subject P corresponding to the position of the light irradiation unit 111 based on the operation signal generated by the signal calculating unit 1223 as data for the time domain and data calculation units 1224 and 1224.

Hereinafter, the distance between the adjacent light irradiation unit 111 and the light receiving unit 112 is 10 mm to 50 mm.

In addition, a distance between the adjacent light irradiation unit 111 and the light receiving unit 112 is 25 mm to 35 mm.

According to a first embodiment of the disclosure, a biosignal imaging apparatus 1 includes: a biosignal measuring apparatus 100 according to the present invention; and an image processing apparatus 200 for obtaining image information on the subject P based on the data for the time domain in the subject P calculated by the signal processing unit 122; The image processing apparatus 200 obtains an extinction coefficient using the wavelength signal shape restored by the signal processing unit 122 and normalizes the interpolation unit 210 to calculate the characteristics of the subject P ; and matching the time domain data of the subject P calculated by the signal processing unit 122 with the characteristics of the subject P calculated by the interpolation unit 210 to match the subject P an imaging unit 220 for obtaining image information about;

A brain image-based brain disease diagnosis system according to the present invention comprises: a biosignal imaging device 1 according to the present invention; a mobile terminal 3 for receiving image information on the subject P through wired/wireless communication with the imaging device 1; and a medical server 4 that receives image information about the subject P through wireless communication with the portable terminal 3, wherein any one of the portable terminal 3 and the medical server 4. One is to generate diagnostic information on the current condition of the patient based on the image information for the subject P, and the diagnostic information is bidirectional between the portable terminal 3 and the medical server 4. It is stored in the medical server 4 while being shared with each other through communication.

The brain imaging-based brain disease diagnosis system according to the present invention further includes a network 2 for sharing the diagnosis information through wireless communication with the mobile terminal 3, and through wired/wireless communication with the network 2 a smart medicine box 5 that shares the diagnosis information, generates medication information for a patient in response to the diagnosis information, and provides it to the network 2; The diagnosis information is shared through wired/wireless communication with the network 2, and monitoring information of the patient including the patient's indoor environment information and the patient's operation information is generated in response to the diagnosis information and provided to the network 2 a patient monitoring unit; and a display unit 9 that shares the diagnosis information through wired/wireless communication with the network 2, generates treatment content in response to the diagnosis information, and provides it to the network 2; and at least any one of the following, wherein the portable terminal 3 provides information provided to the network 2 from at least one of the smart medicine box 5, the patient monitoring unit, and the display unit 9. It is provided to the medical server 4, and the provided information is notified to the patient as the provided information in the original state or the updated information updated by the provided information in the original state through the portable terminal 3, and the medical server 4 matches and stores the provided information and the updated information.

### BRIEF DESCRIPTION OF DRAWINGS

FIG. 1 is a diagram illustrating a biosignal measuring apparatus 100 according to an embodiment of the present invention.

FIG. 2 is a block diagram showing a detailed configuration of a biosignal measuring apparatus 100 according to an embodiment of the present invention.

FIG. 3 is a diagram illustrating a biosignal imaging apparatus 1 according to an embodiment of the present invention.

FIG. 4 is a diagram illustrating a brain image-based brain disease diagnosis system according to an embodiment of the present invention.

### DETAILED DESCRIPTION OF THE INVENTION

Hereinafter, an embodiment of the biosignal measuring apparatus 100, the biosignal imaging apparatus 1, and the brain image-based brain disease diagnosis system according to the present invention will be described with reference to the accompanying drawings. At this time, the present invention is not limited or limited by the examples. In addition, in describing the present invention, detailed descriptions of well-known functions or configurations may be omitted to clarify the gist of the present invention.

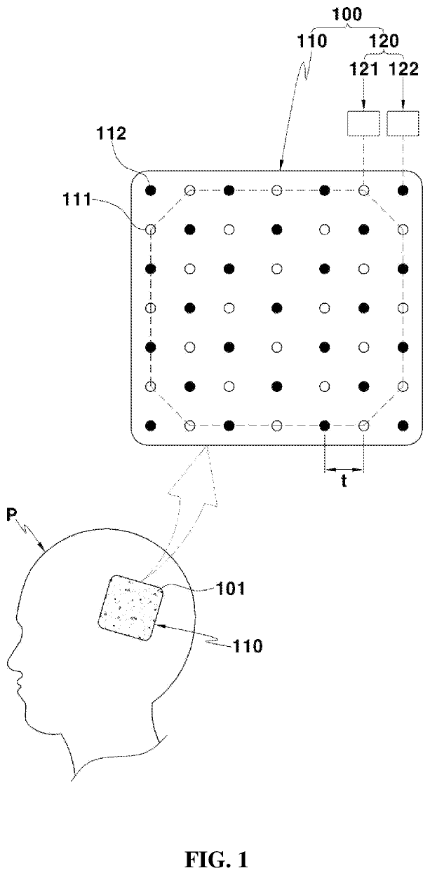

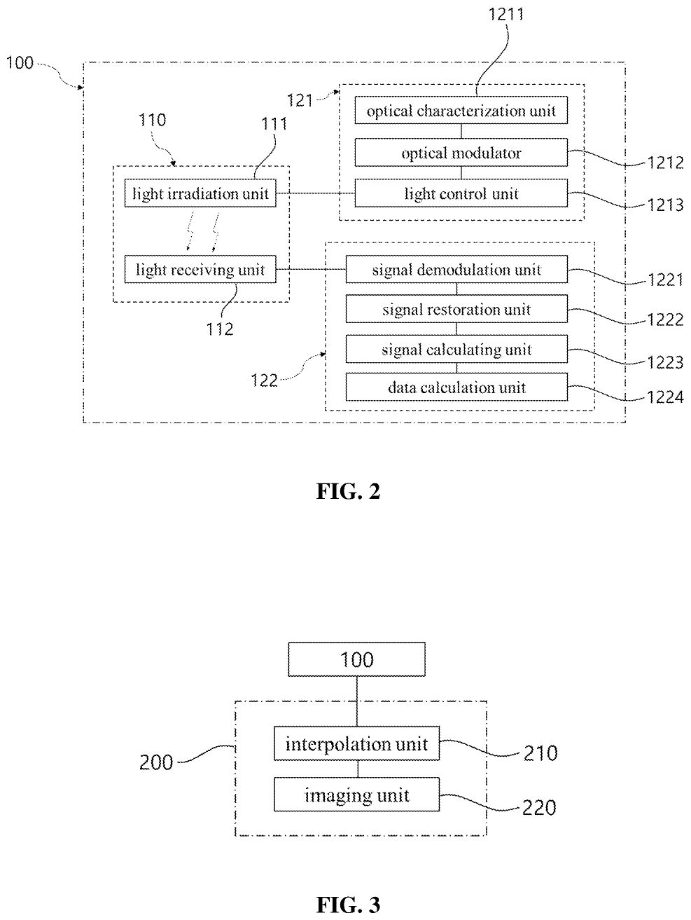

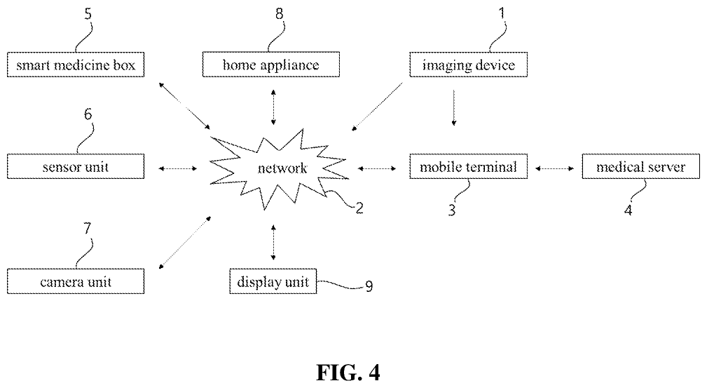

Referring to FIG. 1 and FIG. 2, the biosignal measuring apparatus 100 according to an embodiment of the present invention may include a measuring unit 110 and an operation unit 120.

The measurement unit 110 includes a plurality of light irradiation units 111 for irradiating light to the subject P, and a plurality of light irradiation units 111 for detecting the reflected light signals after the light irradiated from the plurality of light irradiation units 111 is reflected. It may include a light receiving unit 112. Reference numeral 101 denotes a measurement area of the subject.

Hereinafter, the plurality of light irradiating units 111 and the plurality of light receiving units 112 are alternately arranged in a grid shape so that the light irradiating unit 111 and the light receiving unit 112 are alternately arranged at the outermost portion of the measurement unit 110. An imaginary line connecting each light receiving unit 112 located at the outermost portion of the measurement unit 110 has a rectangular shape, and the light receiving unit 112 is disposed at each of the four vertices of the rectangular shape.

In addition, the light emitting unit 111 and the light receiving unit 112 adjacent to each other are spaced apart from each other at equal intervals. At this time, the distance between the adjacent light emitting unit 111 and the light receiving unit 112 is set to be 10 mm to 50 mm. In more detail, the distance between the light emitting unit 111 and the light receiving unit 112 adjacent to each other is set to be 25 mm to 35 mm. When the distance between the light emitting unit 111 and the light receiving unit 112 is out of the set range, the reception sensitivity is lowered and the characteristics of the optical signal detected by the light receiving unit 112 are not exhibited, but the light emitting unit 111 and the light receiving unit 112 When the distance is set within the set range, it is possible to easily check the intensity and phase change of the optical signal detected by the light receiving unit 112 by clarifying the characteristics of the optical signal detected by the light receiving unit 112. In particular, as the setting range is reduced, the optical signal sensitivity of the light receiving unit 112 may be maintained above the preset sensitivity. In particular, when the distance between the adjacent light emitting unit 111 and the light receiving unit 112 is 30 mm, the optical signal sensitivity of the light receiving unit 112 may be maintained at the best state.

The subject P is described as corresponding to the patient's head. Then, the biosignal measuring apparatus 100 according to an embodiment of the present invention measures the cerebral blood vessels inside the subject P according to the depth to calculate the blood flow rate, blood flow velocity, and path length in the subject P. The light irradiator 111 may irradiate at least one of an infrared region, a visible region, and an ultraviolet region as a light source represented by a laser, an LED, or a lamp.

In addition, the light receiving unit 112 may be spaced apart from each other in the upper and lower portions based on one light emitting unit 111, respectively, spaced apart from each other in the left and right portions, or may be spaced apart from each other in the upper and lower portions and the left and right portions. Then, at least three light receiving units 112 are disposed adjacent to one light emitting unit 111 based on one light emitting unit 111.

Then, the light modulated in response to three or more wavelength signals is sequentially irradiated with a time difference in one light irradiator 111 in the measurement unit 110 according to an embodiment of the present invention, and one light irradiator 111 adjacent to the at least three light receiving units 112 may detect an optical signal corresponding to the modulated light sequentially irradiated with a time difference, respectively.

In other words, when three modulated lights are sequentially irradiated with a time difference from one light irradiator 111 and the first modulated light is irradiated at a first time based on one light irradiator 111 , at least three light receiving units When the first light receiving unit 112 of 112 detects the optical signal and the second modulated light is irradiated at a second time based on one light irradiation unit 111 , the second light receiving unit among the at least three light receiving units 112. 112 detects an optical signal, and when the third modulated light is irradiated at the third time based on one light emitting unit 111, the third light receiving unit 112 among the at least three light receiving units 112 receives the optical signal make it detectable.

The arithmetic unit 120 includes a light irradiation control unit 121 for controlling the light signal irradiated from each light irradiation unit 111 and data on the subject P based on the light signal detected by the light receiving unit 112. and a signal processing unit 122 that calculates in the time domain.

The light irradiation control unit controls each light irradiation unit 111 so that light modulated in response to three or more wavelength signals is sequentially irradiated with a time difference based on one light irradiation unit 111.

In more detail, the light irradiation control unit generates three or more modulated signals by modulating the optical characterization unit 1211 that selects three or more wavelength signals and the three or more wavelength signals selected by the optical characterizing unit 1211 with different frequencies, respectively. a light modulator 1212 that controls the light irradiation time from each light irradiation unit 111 so that three or more modulated signals are sequentially irradiated with a time difference based on one light irradiation unit 111.

The three wavelength signals selected by the optical characterization unit 1211 may be, for example, 780 nm, 805 nm, and 830 nm near-infrared light.

In addition, as the three wavelength signals selected by the optical characterization unit 1211, for example, laser light having a wavelength of 355 nm, 532 nm, and 1064 nm may be selected.

The signal processing unit 122 demodulates the optical signals respectively detected by the at least three light receiving units 112 in time series corresponding to the preset positions of the light irradiation units 111, and then restores the demodulated signal to the wavelength signal form before modulation, calculates the blood flow volume, blood flow velocity, and path length in the subject P as data for the time domain based on the restored wavelength signal shape.

In other words, an optical signal detected by some light receiving units 112 corresponding to the modulated light among the plurality of light receiving units 112 is demodulated in time series corresponding to a preset position of the light emitting unit 111, and then the demodulated signal The wave signal form before modulation is restored, and the blood flow volume, blood flow velocity, and path length in the subject P are calculated as data for the time domain based on the restored wave signal form.

In more detail, the signal processing unit 122 demodulates the optical signals respectively detected by the at least three light receiving units 112 in time series corresponding to the preset positions of the light irradiation units 111 to generate a demodulated signal. , a signal restoration unit 1222 that restores each demodulated signal generated by the signal demodulator 1221 to the form of the wavelength signal before modulation, and the waveform signal form restored by the signal restoration unit 1222 to generate an operation signal Based on the signal operation unit 1223 and the operation signal generated by the signal operation unit 1223, the amount of blood flow, the blood flow velocity, and the path length in the subject P corresponding to the position of the light irradiation unit 111 are calculated in the time domain. and a data calculation unit 1224 for calculating.

In other words, the signal demodulation unit 1221 demodulates an optical signal detected by some light receiving units 112 corresponding to a specific modulated signal among the plurality of light receiving units 112 in the time domain with respect to the position of the preset light irradiation unit 111. A demodulated signal can be generated. At this time, in generating the calculation signal in the signal operation unit 1223, according to the diameter of the brain blood vessel of the subject P, when the diameter of the brain blood vessel is smaller than the preset diameter, the data of the largest wave is used, and in the remaining cases, Data of a wavelength smaller than the data of the maximum wavelength may be used.

For example, the signal demodulator 1221 undergoes a demodulation process at least three times in response to the light irradiated from the light irradiator 111, and the signal restorer 1222 undergoes a restoration process at least three times in response to the demodulation process. The calculation unit 1223 may perform one calculation process by combining three restoration processes.

As another example, the signal demodulator 1221 undergoes a demodulation process at least three times in response to the light irradiated from the light irradiator 111, and the signal restorer 1222 collects at least three demodulation processes and undergoes one restoration process, the signal operation unit 1223 may go through one operation process with a signal corresponding to one restoration process.

Hereinafter, the data calculating unit 1224 may calculate the blood flow volume, blood flow velocity, and path length in the subject P corresponding to the position of the light irradiation unit 111 as data for the time domain through various known types.

Hereinafter, the biosignal imaging apparatus 1 according to an embodiment of the present invention will be described.

The biosignal imaging apparatus 1 according to an embodiment of the present invention may include the biosignal measuring apparatus 100 and the image processing apparatus 200 according to an embodiment of the present invention.

The image processing apparatus 200 may acquire image information about the subject P based on the data on the time domain of the subject P calculated by the signal processing unit 122.

In more detail, the image processing apparatus 200 includes an interpolation unit 210 that calculates the characteristics of the subject P by obtaining an extinction coefficient using the wavelength signal shape restored by the signal processing unit 122 and normalizing it; Image information about the subject P is obtained by matching the time domain data of the subject P calculated by the signal processing unit 122 with the characteristics of the subject P calculated by the interpolation unit 210. It may include an imaging unit 220 that

At this time, in normalizing the extinction coefficient in the storage unit, according to the diameter of the cerebral blood vessel of the subject P, when the diameter of the cerebral blood vessel is smaller than the preset diameter, the extinction coefficient of the minimum wavelength is normalized to the extinction coefficient of the maximum wavelength, in other cases, the extinction coefficient of a wavelength greater than the minimum wavelength may be normalized to the extinction coefficient of the maximum wavelength.

Hereinafter, a brain image-based brain disease diagnosis system according to an embodiment of the present invention will be described.

The brain imaging-based brain disease diagnosis system according to an embodiment of the present invention provides a bio-signal imaging apparatus 1 according to an embodiment of the present invention and an imaging device 1 through wired/wireless communication with the subject P. It may include a mobile terminal 3 that receives image information, and a medical server 4 that receives image information about the subject P through wireless communication with the mobile terminal 3.

Then, any one of the portable terminal 3 and the medical server 4 generates diagnostic information on the current state of the patient based on the image information on the subject P. In this case, the diagnosis information may be shared with each other through bidirectional communication between the portable terminal 3 and the medical server 4 and stored in the medical server 4.

The brain image-based brain disease diagnosis system according to an embodiment of the present invention may further include a network 2 for sharing diagnosis information through wireless communication with the portable terminal 3.

In addition, the brain image-based brain disease diagnosis system according to an embodiment of the present invention shares diagnostic information through wired/wireless communication with the network 2, and generates the patient's medication information in response to the diagnosis information and provides it to the network 2. Sharing diagnostic information through wired/wireless communication with the smart medicine box 5 and a display unit 9 that shares diagnostic information through wired/wireless communication with the network 2 and generates treatment content in response to the diagnostic information and provides it to the network 2 may include more.

Then, the portable terminal 3 may provide the medical server 4 with the information provided to the network 2 from at least one of the smart medicine box 5, the patient monitoring unit and the display unit 9. At this time, the provision information is notified to the patient as the provision information in the original state or the updated information of the provision information in the original state through the portable terminal 3. Also, the medical server 4 may match and store the diagnosis information, the provision information, and the update information.

Hereinafter, the network 2 may also be connected to the imaging apparatus 1 through wireless communication. In response to the provided information and updated information, the imaging apparatus 1 may update the image information, and either the portable terminal 3 or the medical server 4 may update and share the diagnosis information.

In more detail, if the smart medicine box 5 is further included, the portable terminal 3 may provide the patient's treatment information provided to the network 2 to the medical server 4 . At this time, the patient's medication information may be reported to the patient in the original state, and the patient's medication information may be updated with the patient's medication record and input to the portable terminal 3.

In addition, the medical server 4 may match and store the diagnosis information, the patient's medication information, and the patient's medication record. Hereinafter, the network 2 may also be connected to the imaging apparatus 1 through wireless communication. At this time, the imaging apparatus 1 may update the image information in response to the patient's medication information and the patient's medication record, and either the portable terminal 3 or the medical server 4 may update and share the diagnostic information.

In addition, if the patient monitoring unit is further included, the portable terminal 3 may provide the patient monitoring information provided to the network 2 to the medical server 4. In this case, the patient's monitoring information may be notified to the patient in the original state, or the patient may be informed of the updated information of the patient's monitoring information in order to change the patient's monitoring information. Such patient monitoring information and changed information for updating the patient monitoring information may be input to the portable terminal 3. In addition, the medical server 4 may match and store the diagnosis information, the monitoring information of the patient, and the updated information of the monitoring information of the patient.

Hereinafter, the network 2 may also be connected to the imaging apparatus 1 through wireless communication. At this time, the imaging apparatus 1 updates the image information in response to the patient's monitoring information and the updated information about the patient's monitoring information, and either the portable terminal 3 or the medical server 4 updates the diagnosis information. can be shared.

The patient monitoring unit is provided in the room where the patient lives, and the patient monitoring unit includes a sensor unit 6 and home appliances 8 that detect the movement of the patient moving in the room, the patient's living pattern, and the indoor environment, and it may include at least one of a camera unit 7 for photographing a moving patient's movement, a patient's living pattern, and an indoor environment, and a wearable unit 6-1 mounted on the patient. Specifically, the wearable unit 6-1 may include at least one of a gyro sensor, an acceleration sensor, and a vibration sensor to detect a dangerous situation including the patient's collapse or fall.

In addition, when the display unit 9 is further included, the display unit 9 may provide the treatment content in the form of virtual reality, augmented reality, or a combination thereof.

The portable terminal 3 may provide the treatment content provided to the network 2 to the medical server 4. In this case, the treatment content may notify the patient in the original state, or notify the patient of updated content including selection of whether to use the treatment content or whether to use the treatment content. Such treatment content and updated content may be input to the portable terminal 3. In addition, the medical server 4 may match and store the diagnosis information, the treatment content, and the updated content.

Hereinafter, the network 2 may also be connected to the imaging apparatus 1 through wireless communication. In this case, the imaging apparatus 1 may update the image information in response to the treatment content and the updated content, and any one of the portable terminal 3 and the medical server 4 may update and share the diagnosis information.

According to the biosignal measuring apparatus 100, the biosignal imaging apparatus 1, and the brain image-based brain disease diagnosis system described above, data on the blood flow volume, blood flow velocity, and path length in the subject P are stored in the time domain. It can be easily calculated with data, and based on this, brain disease diagnosis can be simplified.

In addition, by using three or more wavelengths, in the cerebral blood vessels included in the subject P, it is possible to easily calculate the oxygenation and non-oxygenation state of blood, the hemoglobin state, and the path length of the cerebral blood vessels.

In addition, through the detailed configuration of the light control unit 1213, the light signal irradiated from one light irradiation unit 111 can be specified, and the modulated signal can be generated in response to the blood flow rate, blood flow velocity, and path length in the subject P.

In addition, it is possible to specify the light receiving unit 112 from which the modulated signal is detected through the detailed configuration of the signal processing unit 122, and data for the time domain in response to the blood flow volume, blood flow velocity, and path length in the subject P can be completed.

In addition, by clarifying the characteristics of the optical signal detected by the light receiving unit 112 by limiting the distance between the light emitting unit 111 and the light receiving unit 112, the intensity and phase change of the optical signal detected by the light receiving unit 112 can be easily checked possible.

In addition, by reducing the limitation of the distance between the light emitting unit 111 and the light receiving unit 112, the optical signal sensitivity of the light receiving unit 112 can be maintained in the best state.

In addition, image information on the subject P can be clearly obtained through the detailed configuration of the image processing apparatus 200.

In addition, by analyzing the image information on the subject P, the diagnosis information of the patient can be shared online, and the patient can be treated in various forms.

Although the preferred embodiment of the present invention has been described with reference to the drawings as described above, those skilled in the art may change the present invention in various ways without departing from the spirit and scope of the present invention as set forth in the following claims. may be modified or changed.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A bio-signal measuring device comprises:
a measurement unit comprising a plurality of light irradiation units for irradiating light to the subject and a plurality of light receiving unit for detecting the reflected light signals after the light irradiated from the plurality of light irradiation units, and
The arithmetic unit comprising a light irradiation control unit for controlling the light signal irradiated from each light irradiation unit and a signal processing unit that calculates the data for the subject in the time domain based on the optical signal detected by the light receiving unit,
wherein the plurality of light irradiating units and a plurality of light receiving units are alternately arranged in a grid shape so that the light irradiating unit and the light receiving unit are alternately arranged on the outermost side of the measuring unit,
An imaginary line connecting each light receiving unit located at the outermost portion of the measurement unit has a rectangular shape,
wherein the light receiving unit is disposed at four vertices in the rectangular shape,
wherein the light irradiation control unit includes an optical characterization unit for selecting the three or more wavelength signals; an optical modulator for generating three or more modulated signals by modulating the three or more wavelength signals selected by the optical characterization unit with different frequencies; and a light control unit for controlling the time the light is irradiated from each light irradiation unit so that three or more modulated signals are sequentially irradiated with a time difference based on one light irradiation unit,
wherein the signal processing unit includes: a signal demodulator for generating a demodulated signal by demodulating the optical signals respectively detected by the at least three light receiving units in time series corresponding to the preset positions of the light irradiation units; a signal restoration unit for restoring each demodulated signal generated by the signal demodulation unit to the form of a wavelength signal before modulation; a signal operation unit for generating an operation signal by calculating the shape of the wavelength signal restored by the signal restoration unit; and calculating the blood flow volume, blood flow velocity, and path length in the subject corresponding to the position of the light irradiation unit based on the operation signal generated by the signal calculating unit as data for the time domain and a data calculation unit,
wherein the light irradiation control unit controls each light irradiation unit so that light modulated in response to three or more wavelength signals is sequentially irradiated with a time difference based on one light irradiation unit,
in one light irradiator, light modulated in response to three or more wavelength signals is sequentially irradiated with a time difference,
at least three light receiving units adjacent to one light emitting unit detect an optical signal corresponding to the modulated light sequentially irradiated with a time difference, respectively.
wherein the signal processing unit demodulates the optical signals respectively detected by the at least three light receiving units in time series corresponding to the preset positions of the light irradiation units, and then restores the demodulated signal to the wavelength signal form before modulation. and calculates the blood flow volume, blood flow velocity, and path length in the subject as data for the time domain based on the restored wavelength signal shape.

2. The biosignal measuring device according to claim 1,
the distance between the mutually adjacent light irradiation unit and the light receiving unit characterized in that 10 mm to 50 mm.

3. The biosignal measuring device according to claim 2,
The distance between the mutually adjacent light irradiation unit and the light receiving unit characterized in that 25 mm to 35 mm.

4. A biosignal imaging apparatus comprising:
the biosignal measuring device according to claim 1; and
a image processing apparatus for obtaining image information on the subject based on the data for the time domain in the subject calculated by the signal processing unit;
wherein the image processing apparatus comprising
an interpolation unit for calculating the characteristics of the subject by obtaining an extinction coefficient using the wavelength signal shape restored by the signal processing unit and normalizing it; and
an imaging unit acquiring image information on the data on the time domain of the subject calculated by the signal processing unit and the characteristics of the subject calculated by the interpolation unit are matched to match the subject.

5. A brain imaging-based brain disease diagnosis system comprises:
the biosignal imaging device according to claim 4,
a mobile terminal that receives image information about the subject through wired/wireless communication with the imaging device, and
a medical server receiving image information about the subject through wireless communication with the portable terminal,
any one of the portable terminal and the medical server generates diagnostic information on the current condition of the patient based on the image information on the subject,
wherein the diagnosis information is shared with each other through two-way communication between the portable terminal and the medical server and stored in the medical server.

6. The brain imaging-based brain disease diagnosis system according to claim 5,
wherein the brain imaging-based brain disease diagnosis system further comprising:
a network for sharing the diagnosis information through wireless communication with the mobile terminal,
a smart medicine box that shares the diagnosis information through wired/wireless communication with the network, generates the patient's medication information in response to the diagnosis information, and provides it to the network,
a patient monitoring unit sharing the diagnosis information through wired/wireless communication with the network, and generates patient monitoring information including the patient's indoor environment information and the patient's operation information in response to the diagnosis information to create the network, and
a display unit that shares the diagnosis information through wired/wireless communication with the network, generates treatment content in response to the diagnosis information, and provides it to the network,
wherein the mobile terminal provides the medical server with the information provided to the network from at least one of the smart medicine box, the patient monitoring unit, and the display unit,
wherein the provision information is notified to the patient as the provision information in the original state or the updated information in the original state through the portable terminal,
wherein the medical server matches and stores the provided information and the updated information,
wherein the patient monitoring unit includes a sensor unit that detects a patient's movement, a patient's living pattern, and an indoor environment, a home appliance, a camera unit that captures the indoor environment, and a wearable unit mounted on the patient includes at least one.
